(19) United States
(12) Patent Application Publication
(10) Pub. No.: US 2023/0140717 A1
(43) Pub. Date: May 4, 2023

(54) MORTAL PLURIPOTENT STEM CELLS

(71) Applicant: ACCELERATED BIOSCIENCES CORP., Carlsbad, CA (US)

(72) Inventors: Jau-Nan Lee, Kaohsiung (TW); Tony Tung-Yin LEE, Yakima, WA (US); Yuta LEE, Kaohsiung (TW)

(21) Appl. No.: 17/964,399

(22) Filed: Oct. 12, 2022

Related U.S. Application Data
(63) Continuation of application No. PCT/US21/30686, filed on May 4, 2021.
(60) Provisional application No. 63/020,247, filed on May 5, 2020.

(51) Int. Cl. C12N 5/074; C12N 5/00

(52) U.S. Cl. CPC C12N 5/0607; C12N 5/0031; C12N 2500/44; C12N 2500/38; C12N 2501/115

(57) ABSTRACT

Disclosed herein are mortal pluripotent stem cells produced in vitro and compositions thereof. Disclosed herein are methods of treating a disorder or condition by utilizing the cells disclosed herein. Also disclosed herein are methods of growing cells in culture medium as well as populations of mortal pluripotent stem cells differentiated therefrom.

### CROSS-REFERENCE

This application is a continuation of International Application No. PCT/US2021/30686, filed May 4, 2021, which claims the benefit of U.S. Provisional Application No. 63/020,247, filed May 5, 2020, which application is incorporated herein by reference in its entirety.

### BACKGROUND

There exists a need for novel stem cells for treating various diseases or conditions, as an alternative to overcome certain shortcomings of existing embryonic stem cells and iPS cells.

### INCORPORATION BY REFERENCE

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

### BRIEF SUMMARY

The inventive embodiments provided in this Brief Summary of the Invention are meant to be illustrative only and to provide an overview of selective embodiments disclosed herein. The Brief Summary of the Invention, being illustrative and selective, does not limit the scope of any claim, does not provide the entire scope of inventive embodiments disclosed or contemplated herein, and should not be construed as limiting or constraining the scope of this disclosure or any claimed inventive embodiment.

In some of many aspects, disclosed herein is a population of mortal pluripotent stem cells (MPSCs), wherein the population of MPSCs express HLA-G and insulin, and wherein the population of MPSCs are capable of reaching up to about 89-100 population doublings within 90 days from a start of culturing the MPSCs. In some instances, the population of MPSCs are capable of reaching: about 25-30 population doublings within 12 days, up to about 50-55 population doublings within 30 days, and/or up to about 75-80 population doublings within 63 days, from a start of culturing the MPSCs. In some instances, the population of MPSCs are capable of doubling in about 22-27 hours, for example about 25 hours. In some aspects, disclosed herein is a population of mortal pluripotent stem cells (MPSCs), wherein the population of MPSCs express HLA-G and insulin, and wherein the population of MPSCs are free from a pathogen.

In some cases, the MPSCs disclosed herein are free from a pathogen. In some instances, the MPSCs are free from a bacterium. In some instances, the MPSCs are free from a virus, for example a cytomegalovirus. In some instances, the MPSCs are free from a pathogen selected from the group consisting of EBV (Epstein-Barr virus), HAdV (human adenovirus), HCMV (human cytomegalovirus), Hepatitis A, Hepatitis B, Hepatitis C, HHV 6 (human herpes virus 6), HHV 8 (human herpes virus 8), HIV1 (human immunodeficiency virus 1), HIV2 (human immunodeficiency virus 2), HPV (human papillomavirus), HPV16, HPV18, HSV 1 (herpes simplex 1), HSV 2 (herpes simplex 2), HTLV 1 (human T-lymphotropic virus 1), HTLV 2 (human T-lymphotropic virus 2), VZV (varicella virus), Corynebacterium bovis, Corynebacterium sp. (HAC2), Hantavirus comprising Hantaan, Seoul, or Sin Nombre, LCMV (lymphocytic choriomeningitis virus), Mycoplasma sp., Treponema pallidum, and any combination thereof.

In some instances, the MPSCs disclosed herein, or a population comprising the MPSCs, further express one or more proteins of b-HCG, HSP90, CDX2, FGFR1, pAKT, pCREB1, HLA-A, HLA-B, or HLA-C. In some instances, the population of the MPSCs further express one or more proteins of KIR2DL4, Flt3L, NKp46, TCR, ILT-4, CD49f, CD3, CD4, CD8, CD10, CD11b, CD14, CD16, CD19, CD34, CD38, CD44, CD56, CD90/Thy-1, CD105, CD141, CD146, CD166, or CD107a. In some instances, the population of the MPSCs further express one or more proteins of IL-6, IL-8, MCP-1, CLXL2, PDGF-AA, VEGF, PAI-1, or IL-10. In some instances, at least some of the MPSCs do not express one or more proteins of Ki-67, HSP70, p53, or Syncytin. In some instances, the population of the MPSCs (e.g., at least: 80%, 85%, 90%, 95%, 96%, 97%, 98%, or 99%) express one or more proteins of CD44, CD90, CD105, CD146, CD166, HLA-A, HLA-B, or HLA-C. In some instances, at least some of the MPSCs do not express one or more proteins of CD19, CD45, or HLA-DR. In some instances, more than: 96%, 97%, 98% or 99% of the MPSCs do not express one or more proteins of CD19, CD45, or HLA-DR. In some instances, the population of the MPSCs further express one or more proteins of CD16 or CD56 or a combination thereof. In some instances, at least some of the MPSCs do not express CD3. In some instances, more than: 96%, 97%, 98% or 99% of the MPSCs do not express CD3. In some instances, at least 65% or at least 70% of the population of the MPSCs express the HLA-G. In some instances, the HLA-G comprises HLA-G1, HLA-G2, HLA-G3, HLA-G4, HLA-G5, HLA-G6, or HLA-G7, or any combination thereof. In some instances, the HLA-G comprises HLA-G2, HLA-G4, HLAG-6, or HLA-G7, or any combination thereof. In some instances, the HLA-G comprises HLAG-6, or HLA-G7, or a combination thereof. In some instances, less than 15% (e.g., less than 10%) of the population of the MPSCs express HLA-G1.

In some instances, at least 10% of the population of MPSCs disclosed herein are monoclonal. In some instances, about 13% to about 15% of the population of MPSCs are monoclonal. In some instances, at least about 1×10⁶ MPSCs are present in the population. In some instances, the MPSCs have a stable karyotype as measured by an array-based whole-genome assay. In some instances, the MPSCs experience no chromosomal aberration from population doublings, as measured by an array-based whole-genome assay. In some instances, wherein the MPSCs experience no substantial chromosomal aberration from freezing and thawing, as measured by an array-based whole-genome assay.

In some cases, the present disclosure provides a method of growing the population of MPSCs disclosed herein, comprising seeding a subculture of the MPSCs at a density from about 1,000 to about 5,000 cells/cm² in a culture medium.

In some aspects, disclosed herein is a method of growing a population of mortal pluripotent stem cells (MPSCs), comprising seeding a subculture of the MPSCs at a density from about 1,000 to about 5,000 cells/cm² in a culture medium, and wherein the population of MPSCs express HLA-G and insulin. In some instances, the culture medium is free from an animal component. In some instances, the culture medium is free from serum for example fetal bovine serum. In some instances, the subculture comprises a 3-day subculture. In some instances, the subculture comprises a 4-day subculture. In some instances, the subculture of the MPSCs is at a density from about 2,000 to about 4,000 cells/cm².

In some aspects, disclosed herein is a method of producing cells, comprising contacting a population of MPSCs disclosed herein with an inducing agent. In some instances, the cells are ectodermal cells. In some instances, the cells are mesodermal cells. In some instances, the cells are endodermal cells. In some instances, the cells are pancreatic cells or pancreatic progenitor cells and optionally the inducing agent comprises bFGF (basic fibroblast growth factor) which may further comprise 2-mercaptoethanol and nicotinamide. In some instances, the cells are neural cells or neural progenitor cells and optionally the inducing agent comprises retinoic acid. In some instances, the cells are hepatic cells or hepatic progenitor cells and optionally the inducing agent comprises a fibroblast growth factor (FGF) such as FGF2, a steroid such as dexamethasone, and a cytokine such as oncostatin M, which may further comprise a bone morphogenetic protein (BMP) for example BMP4, and/or a hepatic growth factor. In some instances, the FGF binds to FGFR1 and is FGF1, FGF2, FGF3, FGF4, FGF5, FGF6, FGF8, FGF10, FGF17, FGF19, FGF20, FGF21, FGF22, or FGF23. In some instances, the steroid is a glucocorticoid steroid, e.g., dexamethasone, betamethasone, budesonide, cortisone, hydrocortisone, methylprednisolone, prednisolone, prednisone, or triamcinolone. In some instances, the cytokine is an interleukin 6 group cytokine, e.g., oncostatin M for example a human oncostatin M, interleukin-6, interleukin-11, leukemia inhibitory factor (LIF), ciliary neurotropic factor (CNTF), cardiotrophin-1 (CT-1), and cardiotrophin-like cytokine (CLC). In some instances, the cells are natural killer cells and the inducing agent comprises an FGF, e.g., FGF1, FGF2, FGF3, FGF4, FGF5, FGF6, FGF8, FGF10, FGF17, FGF19, FGF20, FGF21, FGF22, or FGF23.

### BRIEF DESCRIPTION OF THE DRAWINGS

Various aspects of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a line chart showing 3-day growth curves of MPSCs measured by population doublings in a time frame of 90 days.

FIGS. 2A-2D show a whole genome view of a KARYOSTAT™ analysis of four different MPSCs samples at different population doublings.

FIG. 3 shows flow cytometry analysis of MPSCs stained for HLA-G isotypes.

FIG. 4 shows flow cytometry analysis of MPSCs stained with a 4H84 antibody for HLA-G isotypes and with mouse IgG1 for a control.

FIGS. 5A-5D show characterization of MPSCs by expressing specific molecular biomarkers. MPSCs express molecular biomarkers such as (3-hCG, HLA-G, HSP90, and CDX2 (FIG. 5A), but some are negative such as ki67, Syncytin, HSP70, p53 (FIG. 5B). (FIG. 5C) MPSCs express HLA-A,B,C (left panel) and surface and soluble HLA-G detected by 4H84 antibody (right panel) compared to isotype control and unstained cells by FACS analysis. (FIG. 5D) A representative FACS analysis of HLA-G isoforms in MPSCs at the cell surface compared to at the cell surface and intracellular.

FIGS. 6A-6G show expressions of molecular biomarkers of immune cells in MPSCs. By imaging or FACS analysis, MPSCs express various molecular biomarkers of NK cells (FIG. 6A), T cells (FIG. 6B), dendritic cells (FIG. 6C and FIG. 6D), macrophages (FIG. 6E), and stem cell progenitors (FIG. 6F and FIG. 6G). The specific biomarkers are indicated on the images or plots.

### DETAILED DESCRIPTION

Disclosed herein are novel unique mortal pluripotent stem cells (MPSCs) produced in vitro, compositions thereof, and uses thereof in generating differentiated cells of various phenotypes (e.g., pancreatic, neural, hepatic, immunoregulatory, or natural killer cell phenotype) or treating disorders (e.g., diabetes, neural loss or degeneration, liver diseases, cancers, inflammations, viral infections, or autoimmune diseases) or improving conditions (e.g., skin conditions). The MPSCs are distinct from previous trophoblast stem cells and have advantages including but not limited to: fast and scalable population doublings; demonstrated pathogen-free profile; being highly immune privileged and suitable for transplantation; having exceptional chromosomal stability, for example possessing stable karyotype at least to 71 population doublings; and producing robust secretome rich with cytokine, chemokines, and exosomes. The MPSCs are distinct from embryonic stem cells, and are ethically sourced and cultured. Although the MPSCs are mortal (e.g., having definite proliferation capacities), they are capable of reaching population doubling much faster than embryonic stem cells and iPS cells. Unlike the cells from placenta, umbilical cord, or bone marrow, the MPSCs are pluripotent and capable of differentiating or maturing into three primary group of cells that form a human being: ectoderm (giving rising to the skin, neurons, and nervous system), endoderm (forming the gastrointestinal and respiratory tracts, endocrine glands, liver or hepatocyte-like cells, and pancreas or pancreatic cells), and mesoderm (forming bone (osteocytes), adipose, cartilage (chondrocytes), most of the circulatory system, muscles, connective tissue, immune cells, and more). Furthermore, the MPSCs are non-tumorigenic, e.g., not inducing tumor or teratoma, as demonstrated in the studies of immune competent rats.

The details of one or more inventive embodiments are set forth in the accompanying drawings, the claims, and the description herein. Other features, objects, and advantages of the inventive embodiments disclosed and contemplated herein can be combined with any other embodiment unless explicitly excluded.

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range, e.g., ±15% of a referenced numeral value. About also includes the exact amount, for example “about 5 μL” means “about 5 μL” and also “5 μL.” Generally, the term “about” includes an amount that would be expected to be within experimental error.

The terms “treating,” “treatment,” and the like are used herein to mean obtaining a desired pharmacologic and/or physiologic effect. In some instances, an individual (e.g., an individual suspected to be suffering from and/or genetically pre-disposed to a liver-associated disease or disorder is treated prophylactically with a preparation of cells described herein and such prophylactic treatment completely or partially prevents a liver-associated disease or disorder or sign or symptom thereof. In some instances, an individual is treated therapeutically (e.g., when an individual is suffering from a liver-associated disease or disorder), such therapeutic treatment causes a partial or complete cure for the disease or disorder and/or reverses an adverse effect attributable to the disease or disorder and/or stabilizes the disease or disorder and/or delays progression of the disease or disorder and/or causes regression of the disease or disorder.

Administration (e.g., transplantation) of cells disclosed herein to an area in need of treatment is achieved by, for example and not by way of limitation, local infusion during surgery, by injection, by means of a catheter, or by means of an implant, said implant being of a porous, non-porous, or gelatinous material, including membranes, such as sialastic membranes, or fibers.

“Transplanting” a composition into a mammal refers to introducing the composition into the body of the mammal by any method established in the art. The composition being introduced is the “transplant”, and the mammal is the “recipient”. The transplant and the recipient can be syngeneic, allogeneic or xenogeneic. Further, the transplantation can be an autologous transplantation.

The term “isolated,” when used in relation to a cell or a population of cells, refers to the state of the cell or population of cells being separate from and not present in a host organism, from which the cell or the population of cells may be derived. In some instances, an isolated cell is in contact with other cells that are isolated or derived from the same host organism. In some instances, an isolated cell is purified and separate from any other cells. In some instances, an isolated cell is derived in vitro from a stem cell.

An “effective amount” is an amount of a therapeutic agent sufficient to achieve the intended purpose. An effective amount of a composition to treat or ameliorate a disorder is an amount of the composition sufficient to reduce or remove the symptoms of the disorder.

The section headings used herein are for organizational purposes only and are not to be construed as limiting the subject matter described.

### Cells and Compositions

In some of many aspects, disclosed herein is a population of mortal pluripotent stem cells (MPSCs), wherein the population of MPSCs express HLA-G and insulin, and wherein the population of MPSCs are capable of reaching up to about 89-100 population doublings within 90 days from a start of culturing the MPSCs. In some instances, the population of MPSCs are capable of reaching: about 25-30 population doublings within 12 days, up to about 50-55 population doublings within 30 days, and/or up to about 75-80 population doublings within 63 days, from a start of culturing the MPSCs. In some instances, the population of MPSCs are capable of doubling in about 22-27 hours, for example about 25 hours. In some aspects, disclosed herein is a population of mortal pluripotent stem cells (MPSCs), wherein the population of MPSCs express HLA-G and insulin, and wherein the population of MPSCs are free from a pathogen. In some instances, the MPSC lacks expression of p53, Syncytin, Ki67, heat shock protein 70 (HSP70), or any combination thereof. In some instances, the MPSC is a human cell. In some instances, the MPSC is originated from a rodent, rabbit, cow, sheep, pig, dog, cat, monkey, or ape.

In some cases, the MPSCs disclosed herein are free from a pathogen. In some instances, the MPSCs are free from a bacterium. In some instances, the MPSCs are free from a virus, for example a cytomegalovirus. In some instances, the MPSCs are free from a pathogen selected from the group consisting of EBV (Epstein-Barr virus), HAdV (human adenovirus), HCMV (human cytomegalovirus), Hepatitis A, Hepatitis B, Hepatitis C, HHV 6 (human herpes virus 6), HEW 8 (human herpes virus 8), HIV1 (human immunodeficiency virus 1), HIV2 (human immunodeficiency virus 2), HPV (human papillomavirus), HPV16, HPV18, HSV 1 (herpes simplex 1), HSV 2 (herpes simplex 2), HTLV 1 (human T-lymphotropic virus 1), HTLV 2 (human T-lymphotropic virus 2), VZV (varicella virus), Corynebacterium bovis, Corynebacterium sp. (HAC2), Hantavirus comprising Hantaan, Seoul, or Sin Nombre, LCMV (lymphocytic choriomeningitis virus), Mycoplasma sp., Treponema pallidum, and any combination thereof.

In some instances, the MPSCs disclosed herein, or a population comprising the MPSCs, further express one or more proteins of b-HCG, HSP90, CDX2, FGFR1, pAKT, pCREB1, HLA-A, HLA-B, or HLA-C. In some instances, the population of the MPSCs further express one or more proteins of KIR2DL4, Flt3L, NKp46, TCR, ILT-4, CD49f, CD3, CD4, CD8, CD10, CD11b, CD14, CD16, CD19, CD34, CD38, CD44, CD56, CD90/Thy-1, CD105, CD141, CD146, CD166, or CD107a. In some instances, the population of the MPSCs further express one or more proteins of IL-6, IL-8, MCP-1, CLXL2, PDGF-AA, VEGF, PAI-1, or IL-10. In some instances, at least some of the MPSCs do not express one or more proteins of Ki-67, HSP70, p53, or Syncytin. In some instances, the population of the MPSCs (e.g., at least: 80%, 85%, 90%, 95%, 96%, 97%, 98%, or 99%) express one or more proteins of CD44, CD90, CD105, CD146, CD166, HLA-A, HLA-B, or HLA-C. In some instances, at least some of the MPSCs do not express one or more proteins of CD19, CD45, or HLA-DR. In some instances, more than: 96%, 97%, 98% or 99% of the MPSCs do not express one or more proteins of CD19, CD45, or HLA-DR. In some instances, the population of the MPSCs further express one or more proteins of CD16 or CD56 or a combination thereof. In some instances, at least some of the MPSCs do not express CD3. In some instances, more than: 96%, 97%, 98% or 99% of the MPSCs do not express CD3. In some instances, at least 65% or at least 70% of the population of the MPSCs express the HLA-G. In some instances, the HLA-G comprises HLA-G1, HLA-G2, HLA-G3, HLA-G4, HLA-G5, HLA-G6, or HLA-G7, or any combination thereof. In some instances, the HLA-G comprises HLA-G2, HLA-G4, HLAG-6, or HLA-G7, or any combination thereof. In some instances, the HLA-G comprises HLAG-6, or HLA-G7, or a combination thereof. In some instances, less than 15% (e.g., less than 10%) of the population of the MPSCs express HLA-G1.

In some instances, at least 10% of the population of MPSCs disclosed herein are monoclonal. In some instances, about 13% to about 15% of the population of MPSCs are monoclonal. In some instances, at least about 1×10⁶ MPSCs are present in the population. In some instances, the MPSCs have a stable karyotype as measured by an array-based whole-genome assay. In some instances, the MPSCs experience no chromosomal aberration from population doublings, as measured by an array-based whole-genome assay. In some instances, wherein the MPSCs experience no substantial chromosomal aberration from freezing and thawing, as measured by an array-based whole-genome assay.

In some cases, the cells provided herein, e.g., MPSCs, are genetically modified. In some instances, the cell is genetically modified to express an exogenous gene, e.g., transgene. The term “transgene” and its grammatical equivalents as used herein can refer to a gene or genetic material that is transferred into an organism. For example, a transgene can be a stretch or segment of DNA containing a gene that is introduced into an organism. When a transgene is transferred into an organism, the organism is then referred to as a transgenic organism. A transgene can retain its ability to produce RNA or polypeptides (e.g., proteins) in a transgenic organism. A transgene can be composed of different nucleic acids, for example RNA or DNA. A transgene may encode for an engineered T cell receptor, for example a TCR transgene. A transgene may comprise a TCR sequence. A transgene can comprise an oncogene. A transgene can comprise an immune oncogene. A transgene can comprise recombination arms. A transgene can comprise engineered sites. In some instances, a transgene is an oncogene. In some instances, a transgene is an immune oncogene. In some instances, a transgene is a tumor suppressor gene. In some instances, a transgene encodes a protein that directly or indirectly promotes proteolysis. In some instances, a transgene is an oncolytic gene. In some instances, a transgene can aid a lymphocyte in targeting a tumor cell. In some instances, a transgene is a T cell enhancer gene. In some instances, a transgene is an oncolytic virus gene. In some instances, a transgene inhibits tumor cell growth. In some instances, a transgene is an anti-cancer receptor. In some instances, a transgene is an anti-angiogenic factor. In some instances, a transgene is a cytotoxic gene. Exemplary transgenes include, but are not limited to, CD28, inducible co-stimulator (ICOS), CD27, 4-1BB (CD137), ICOS-L, CD70, 4-1BBL, Signal 3, a cytokine such as IL-2, IL-7, IL-12, IL-15, IL-21, ICAM-1 (CD54), LFA-3 (CD58), HLA class I genes, B7, CD80, CD83, CD86, CD32, CD64, 4-1BBL, CD3, CD1d, CD2, membrane-bound IL-15, membrane-bound IL-17, membrane-bound IL-21, membrane-bound IL-2, truncated CD19, VEGF, Caspase, a chemokine, or one or more genes encoding an antibody (e.g., a monoclonal antibody) to any of the above, or any combination thereof. In some instances, a transgene encodes a protein involved in cell or tissue repair (e.g., proteins associated with DNA repair, the immune response (e.g., interferons and interleukins), and structural proteins). In some instances, a transgene encodes a growth factor receptor. In some instances, a MPSC as described herein comprises a transgene coding for a TCR, a B cell receptor (BCR), a chimeric antigen receptor (CAR), or any combination thereof. In some instances, a MPSC as described herein comprises a transgene coding for an oncogene receptor.

In some cases, a composition comprising cells disclosed herein is formulated as a pharmaceutical composition for intravenous administration to a mammal, including a human. In some instances, compositions for intravenous administration are solutions in sterile tonic aqueous buffer. Where necessary, the composition also includes a local anesthetic to ameliorate any pain at the site of the injection. Where the composition is to be administered by infusion, it can be dispensed with an infusion bottle containing sterile pharmaceutical grade water or saline. Where the composition is administered by injection, an ampoule of sterile water for injection or saline can be provided so that the ingredients are mixed prior to administration.

In one aspect, disclosed herein is a composition (e.g., pharmaceutical composition) comprising a cell disclosed herein. In some instances, the compositions further comprise a pharmaceutically acceptable carrier or excipient. Such a carrier includes, but is not limited to, saline, buffered saline, dextrose, water, and combinations thereof. In other examples, a colloidal dispersion system is used. Colloidal dispersion systems include macromolecule complexes, nanocapsules, microspheres, beads, and lipid-based systems, including oil-in-water emulsions, micelles, mixed micelles, and liposomes.

### Methods of Use

In some aspects, disclosed herein is a method of producing cells, comprising contacting the population of MPSCs disclosed herein with an inducing agent. In some instances, the cells are ectodermal cells. In some instances, the cells are mesodermal cells. In some instances, the cells are endodermal cells. In some instances, the cells are pancreatic cells or pancreatic progenitor cells and optionally the inducing agent comprises bFGF (basic fibroblast growth factor) which may further comprise 2-mercaptoethanol and nicotinamide. In some instances, the cells are neural cells or neural progenitor cells and optionally the inducing agent comprises retinoic acid. In some instances, the cells are hepatic cells or hepatic progenitor cells and optionally the inducing agent comprises a fibroblast growth factor (FGF) such as FGF2, a steroid such as dexamethasone, and a cytokine such as oncostatin M, which may further comprise a bone morphogenetic protein (BMP) for example BMP4, and/or a hepatic growth factor. In some instances, the FGF binds to FGFR1 and is FGF1, FGF2, FGF3, FGF4, FGF5, FGF6, FGF8, FGF10, FGF17, FGF19, FGF20, FGF21, FGF22, or FGF23. In some instances, the steroid is a glucocorticoid steroid, e.g., dexamethasone, betamethasone, budesonide, cortisone, hydrocortisone, methylprednisolone, prednisolone, prednisone, or triamcinolone. In some instances, the cytokine is an interleukin 6 group cytokine, e.g., oncostatin M for example a human oncostatin M, interleukin-6, interleukin-11, leukemia inhibitory factor (LIF), ciliary neurotropic factor (CNTF), cardiotrophin-1 (CT-1), and cardiotrophin-like cytokine (CLC). In some instances, the cells are natural killer cells and the inducing agent comprises an FGF, e.g., FGF1, FGF2, FGF3, FGF4, FGF5, FGF6, FGF8, FGF10, FGF17, FGF19, FGF20, FGF21, FGF22, or FGF23. In some instances, MPSCs can differentiate to neural progenitor cells in 1 day with 1-step protocol, compared to a 30-day (or 3-4 weeks) and 4-step differentiation of embryonic stem cells or iPS cells. In some instances, MPSCs can differentiate into insulin-producing pancreatic progenitor cells in 1 day with 1-step protocol, compared to 8 to 15-day and 4 to 5-step differentiation of embryonic stem cells or iPS cells. In some instances, MPSCs differentiate to hepatocyte-like cells in 6 days with a 2 step protocol, compared to 12 to 21-days and 3-step differentiation of embryonic stem cells or iPS cells.

In some cases, a cell disclosed herein is administered to the subject intravenously, subcutaneously, percutaneously, inhalationally, orally, intramuscularly, or intratumorally. In some instances, the subject is a mammal. In some instances, the subject is a primate. In some instances, the subject is a human.

In some cases, disclosed herein is a method for killing an antigen-bearing target cell, comprising administering to a subject in need thereof a cell disclosed herein. In some instances, the antigen-bearing target cell is a cancer cell. In some instances, the cancer cell is a solid tumor cell. In some instances, the cancer cell is a blood cancer cell. In some instances, the cancer cell comprises bladder cancer cell, bone cancer cell, brain cancer cell, breast cancer cell, carcinoma of cervix, colorectal cancer cell, esophageal cancer cell, gastrointestinal cancer cell, hematopoietic malignancy, head and neck squamous cell carcinoma, leukemia, liver cancer cell, lung cancer cell, lymphoma, myeloma, nasal cancer cell, nasopharyngeal cancer cell, oral cancer cell, oropharyngeal cancer cell, ovarian cancer cell, prostate cancer cell, sarcoma, stomach cancer cell, melanoma, thyroid cancer cell, or any combination thereof. In some instances, the antigen-bearing target cell is a pathogen. In some instances, the pathogen comprises virus, bacterium, protozoa, prion, fungus, or any combination thereof. In some instances, the method kills at least about 5%, at least about 10%, at least about 20%, at least about 50%, at least about 80%, at least about 90%, at least about 95%, at least about 99%, or about 100% of a population of antigen-bearing target cells.

In some cases, disclosed herein is a method for downregulating an inflammatory pathway, comprising administering to a subject in need thereof a cell disclosed herein. In some instances, the method treats a disease or condition that comprises transplant rejection, infection, endotoxic shock associated with infection, arthritis, rheumatoid arthritis, psoriatic arthritis, systemic onset juvenile idiopathic arthritis (JIA), inflammatory bowel disease (IBD), systemic lupus erythematosus (SLE), asthma, pelvic inflammatory disease, Alzheimer's Disease, Crohn's disease, ulcerative colitis, irritable bowel syndrome, multiple sclerosis, ankylosing spondylitis, dermatomyositis, uveitis, Peyronie's Disease, coeliac disease, gallbladder disease, Pilonidal disease, peritonitis, psoriasis, vasculitis, surgical adhesions, stroke, Type I diabetes, Lyme arthritis, meningoencephalitis, immune mediated inflammatory disorders of the central and peripheral nervous system, pancreatitis, trauma from surgery, graft-versus-host disease, heart disease, bone resorption, burns patients, myocardial infarction, Paget's disease, osteoporosis, sepsis, liver or lung fibrosis, periodontitis, or hypochlorhydria. In some instances, the method treats an autoimmune disease that comprises Type I diabetes, multiple sclerosis, systemic lupus erythematosus, Sjogren's syndrome, scleroderma, polymyositis, chronic active hepatitis, mixed connective tissue disease, primary biliary cirrhosis, pernicious anemia, autoimmune thyroiditis, idiopathic Addison's disease, vitiligo, gluten-sensitive enteropathy, Graves' disease, myasthenia gravis, autoimmune neutropenia, idiopathic thrombocytopenia purpura, rheumatoid arthritis, cirrhosis, pemphigus vulgaris, autoimmune infertility, Goodpasture's disease, bullous pemphigoid, discoid lupus, ulcerative colitis, dense deposit disease, inflammatory bowel disease, or psoriasis. In some instances, the method treats Type 1 diabetes. In some instances, the method ameliorates transplant rejection.

In another aspect, disclosed herein is a method of treating a condition in a subject, comprising administering to a subject a pharmaceutical composition that comprises a cell herein, in an amount effective for the cells to engraft to the subject (e.g., to the subject's liver). In some instances, the cells are administered in a pharmaceutically acceptable carrier. In some instances, the pharmaceutically acceptable carrier comprises a saline for example a phosphate buffer saline, or fetal bovine serum. In some instances, the cells are administered in a suspension containing about 1×10⁶ to about 100×10⁶ cells per ml, about 1×10⁶ to about 250×10⁶ cells per ml, about 1×10⁶ to about 500×10⁶ cells per ml, or about 10×10⁶ to about 40×10⁶ cells per ml. In some instances, the cells are administered in a volume of about: 1-5 ml, 1-10 ml, 1-50 ml, 1-100 ml, or 10-150 ml. In some instances, the subject is a human. In some instances, the administering comprises an injection, e.g., intravenous injection. In some instances, the injection is administered at a hepatic vein. In some instances, the injection is administered at a hepatic artery. In some instances, the condition is a liver-associated disease or disorder, e.g., acute liver disease. In some instances, the condition is a liver failure. In some instances, the liver-associated disease or disorder comprises alagille syndrome, alpha 1 anti-trypsin deficiency, autoimmune hepatitis, benign liver tumors, biliary atresia, cirrhosis, cystic disease of the liver, fatty liver disease including alcohol-related liver disease and non-alcohol fatty liver disease (NAFLD), galactosemia, gallstones, Gilbert's Syndrome, hemochromatosis, liver cysts, liver cancer, liver disease in pregnancy (optionally, acute fatty liver of pregnancy, intrahepatic cholestasis of pregnancy, preeclampsia, or HELLP Syndrome (hemolysis, elevated liver tests, low platelets)), neonatal hepatitis, primary biliary cirrhosis, primary sclerosing cholangitis, porphyria, Reye's Syndrome, sarcoidosis, toxic hepatitis, type 1 glycogen storage disease, tyrosinemia, viral hepatitis, Wilson disease, or any combination thereof.

Modes of administration of cells disclosed herein include, but are not limited to, systemic intravenous injection and injection directly to the intended site of activity (e.g., endoscopic retrograde injection). The preparation can be administered by any convenient route, for example, by infusion or bolus injection, and can be administered together with other biologically active agents. In some instances, the administration is systemic localized administration.

In some aspects, provided herein are compositions and methods for transplanting cells disclosed herein to subjects. In some instances, the subject is injected by the cells (e.g., intravenously, intramuscularly, transdermally, endoscopic retrograde injection, or intraperitoneally). In some instances, the subject is not treated with an immunosuppressive agent prior to the transplanting. In some instances, the method further comprises treating the patient with an immunosuppressive agent, e.g., FK-506, cyclosporin, or GAD65 antibodies.

In some instances, cells described herein are delivered to a targeted site (e.g., a defect section of the liver) by a delivery system suitable for targeting cells to a particular tissue. For example, the cells are encapsulated in a delivery vehicle that allows for the slow release of the cell(s) at the targeted site. The delivery vehicle is modified such that it is specifically targeted to a particular tissue. The surface of the targeted delivery system is modified in a variety of ways. In the case of a liposomal-targeted delivery system, lipid groups are incorporated into the lipid bilayer of the liposome in order to maintain the targeting ligand in stable association with the liposomal bilayer.

The administration of cells described herein is optionally tailored to an individual, by: (1) increasing or decreasing the amount cells injected; (2) varying the number of injections; or (3) varying the method of delivery of the cells.

### Detection Methods

Methods for determining the expression or presence of biomarkers described supra are well known in the art, and can be measured, for example, by flow cytometry, immunohistochemistry, Western Blot, immunoprecipitation, magnetic bead selection, and quantification of cells expressing either of these cell surface markers. Biomarker RNA expression levels could be measured by RT-PCR, Qt-PCR, microarray, Northern blot, or other similar technologies.

By “detecting expression” or detecting “expression levels” is intended for determining the expression level or presence of a biomarker protein or gene in the biological sample. Thus, “detecting expression” encompasses instances where a biomarker is determined not to be expressed, not to be detectably expressed, expressed at a low level, expressed at a normal level, or overexpressed.

In some instances, the expression or presence of a biomarker described herein is determined at a nucleic acid level, using, for example, immunohistochemistry techniques or nucleic acid-based techniques such as in situ hybridization and RT-PCR. In some instances, the expression or presence of one or more biomarkers is carried out by a means for nucleic acid amplification, a means for nucleic acid sequencing, a means utilizing a nucleic acid microarray (DNA and RNA), or a means for in situ hybridization using specifically labeled probes.

In some instances, the determining the expression or presence of a biomarker is carried out through gel electrophoresis. In some instances, the determination is carried out through transfer to a membrane and hybridization with a specific probe. In some instances, the determining the expression or presence of a biomarker is carried out by a diagnostic imaging technique. In some instances, the determining the expression or presence of a biomarker is carried out by a detectable solid substrate. In some instances, the detectable solid substrate is paramagnetic nanoparticles functionalized with antibodies.

In some instances, the expression or presence of a biomarker is at an RNA (e.g. mRNA) level. In some instances, techniques that detect RNA (e.g. mRNA) level include, but are not limited to, Southern or Northern analyses, polymerase chain reaction analyses and probe arrays.

One method for the detection of mRNA levels involves contacting the isolated mRNA with a nucleic acid molecule (probe) that hybridize to the mRNA encoded by the gene being detected. The nucleic acid probe comprises of, for example, a full-length cDNA, or a portion thereof, such as an oligonucleotide of at least 7, 15, 30, 50, 100, 250 or 500 nucleotides in length and sufficient to specifically hybridize under stringent conditions to an mRNA or genomic DNA encoding a biomarker described herein. Hybridization of an mRNA with the probe indicates that the biomarker or other target protein of interest is being expressed.

In some instances, the mRNA is immobilized on a solid surface and contacted with a probe, for example by running the isolated mRNA on an agarose gel and transferring the mRNA from the gel to a membrane, such as nitrocellulose. In some instances, the probe(s) are immobilized on a solid surface and the mRNA is contacted with the probe(s), for example, in a gene chip array. A skilled artisan readily adapts known mRNA detection methods for use in detecting the level of mRNA encoding the biomarkers or other proteins of interest.

An alternative method for determining the level of an mRNA of interest in a sample involves the process of nucleic acid amplification, e.g., by RT-PCR, ligase chain reaction, self-sustained sequence replication, transcriptional amplification system, Q-Beta Replicase, rolling circle replication or any other nucleic acid amplification method, followed by the detection of the amplified molecules using techniques well known to those of skill in the art. These detection schemes are especially useful for the detection of nucleic acid molecules if such molecules are present in very low numbers. In some instances, biomarker expression is assessed by quantitative fluorogenic RT-PCR (e.g., the TAQMAN® System).

Expression levels of an RNA of interest are monitored using a membrane blot (such as used in hybridization analysis such as Northern, dot, and the like), or microwells, sample tubes, gels, beads or fibers (or any solid support comprising bound nucleic acids). The detection of expression also comprises using nucleic acid probes in solution.

In some instances, microarrays are used to determine expression or presence of one or more biomarkers. Nucleic acid microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning Hybridization intensities for each probe on the array are determined and converted to a quantitative value representing relative gene expression levels. High-density oligonucleotide arrays are particularly useful for determining the gene expression profile for a large number of RNAs in a sample.

In some instances, an array is fabricated on a surface of virtually any shape or even a multiplicity of surfaces. In some instances, an array is a planar array surface. In some instances, arrays include peptides or nucleic acids on beads, gels, polymeric surfaces, fibers such as fiber optics, glass or any other appropriate substrate. In some instances, arrays are packaged in such a manner as to allow for diagnostics or other manipulation of an all-inclusive device.

In some instances, the expression or presence of a biomarker described herein is determined at a protein level, using, for example, antibodies that are directed against specific biomarker proteins. These antibodies are used in various methods such as Western blot, ELISA, multiplexing technologies, immunoprecipitation, or immunohistochemistry techniques. In some instances, detection of biomarkers is accomplished by ELISA. In some instances, detection of biomarkers is accomplished by electrochemiluminescence (ECL).

Any means for specifically identifying and quantifying a biomarker in the biological sample is contemplated. Thus, in some instances, expression level of a biomarker protein of interest in a biological sample is detected by means of a binding protein capable of interacting specifically with that biomarker protein or a biologically active variant thereof. In some instances, labeled antibodies, binding portions thereof, or other binding partners are used. The word “label” when used herein refers to a detectable compound or composition that is conjugated directly or indirectly to the antibody so as to generate a “labeled” antibody. In some instances, the label is detectable by itself (e.g., radioisotope labels or fluorescent labels) or, in the case of an enzymatic label, catalyzes chemical alteration of a substrate compound or composition that is detectable.

The antibodies for detection of a biomarker protein are either monoclonal or polyclonal in origin, or are synthetically or recombinantly produced. The amount of complexed protein, for example, the amount of biomarker protein associated with the binding protein, for example, an antibody that specifically binds to the biomarker protein, is determined using standard protein detection methodologies known to those of skill in the art. A detailed review of immunological assay design, theory and protocols are found in numerous texts in the art.

The choice of marker used to label the antibodies will vary depending upon the application. However, the choice of the marker is readily determinable to one skilled in the art. These labeled antibodies are used in immunoassays as well as in histological applications to detect the presence of any biomarker or protein of interest. The labeled antibodies are either polyclonal or monoclonal. Further, the antibodies for use in detecting a protein of interest are labeled with a radioactive atom, an enzyme, a chromophoric or fluorescent moiety, or a colorimetric tag as described elsewhere herein. The choice of tagging label also will depend on the detection limitations desired. Enzyme assays (e.g., ELISAs) typically allow detection of a colored product formed by interaction of the enzyme-tagged complex with an enzyme substrate. Radionuclides that serve as detectable labels include, for example, 1-131, 1-123, 1-125, Y-90, Re-188, Re-186, At-211, Cu-67, Bi-212, and Pd-109. Examples of enzymes that serve as detectable labels include, but are not limited to, horseradish peroxidase, alkaline phosphatase, beta-galactosidase, and glucose-6-phosphate dehydrogenase. Chromophoric moieties include, but are not limited to, fluorescein and rhodamine. The antibodies are conjugated to these labels by methods known in the art. For example, enzymes and chromophoric molecules are conjugated to the antibodies by means of coupling agents, such as dialdehydes, carbodiimides, dimaleimides, and the like. Alternatively, conjugation occurs through a ligand-receptor pair. Examples of suitable ligand-receptor pairs are biotin-avidin or biotin-streptavidin, and antibody-antigen.

In some instances, expression or presence of one or more biomarkers or other proteins of interest within a biological sample is determined by radioimmunoassays or enzyme-linked immunoassays (ELISAs), competitive binding enzyme-linked immunoassays, dot blot, Western blot, chromatography such as high performance liquid chromatography (HPLC), or other assays known in the art. Thus, the detection assays involve steps such as, but not limited to, immunoblotting, immunodiffusion, immunoelectrophoresis, or immunoprecipitation.

### Methods of Obtaining Cells

In some cases, a non-embryonic mammalian stem cell (e.g., a trophoblast stem cell) can be a source cell to make mortal pluripotent stem cells (MPSCs) disclosed herein. In some instances, the mammalian stem cells are isolated from amniotic fluid, amniotic membrane, Wharton's jelly, chorionic villi, or ectopic pregnancy, in a manner that is not disturbing nor destructive to an embryo.

In some instances, the MPSC is obtained in a culture medium free from an antibiotic, for instance, penicillin, streptomycin, or any combination thereof. In some instances, the culture medium for obtaining the mammalian stem cell is free from retinoic acid. In some instances, the culture medium obtaining and/or passaging the mammalian stem cell is free from mercaptoethanol, nicotinamide, or a combination thereof. In some instances, the culture medium obtaining and/or passaging the mammalian stem cell is free from dexamethasone, recombinant human oncostatin M, BMP4, HGF, or any combination thereof. In some instances, the culture medium obtaining and/or passaging the mammalian stem cell is xeno-free, e.g., free from an animal component. In some instances, the culture medium obtaining and/or passaging the mammalian stem cell is free from a human derived component and an animal-derived component, e.g., a chemically defined medium. In some instances, the culture medium obtaining and/or passaging the mammalian stem cell is free from a serum. In some instances, the culture medium obtaining and/or passaging the mammalian stem cell is free from fetal bovine serum.

In some cases, the present disclosure provides a method of growing the population of MPSCs disclosed herein, comprising seeding a subculture of the MPSCs at a density from about 1,000 to about 5,000 cells/cm² in a culture medium.

In some aspects, disclosed herein is a method of growing a population of mortal pluripotent stem cells (MPSCs), comprising seeding a subculture of the MPSCs at a density from about 1,000 to about 5,000 cells/cm² in a culture medium, and wherein the population of MPSCs express HLA-G and insulin. In some instances, the culture medium is free from an animal component. In some instances, the culture medium is free from serum for example fetal bovine serum. In some instances, the subculture comprises a 3-day subculture. In some instances, the subculture comprises a 4-day subculture. In some instances, the subculture of the MPSCs is at a density from about 2,000 to about 4,000 cells/cm².

In some instances, the mammalian stem cell can be isolated from amniocentesis biopsies or from amniotic fluid. In one instance, amniocentesis can be a procedure used to obtain a small sample of the amniotic fluid that surrounds the fetus during pregnancy. In one instance, an amniocentesis can be offered to women between the 15th and 20th weeks of pregnancy who are at increased risk for chromosome abnormalities, e.g., women who are over 35 years of age at delivery, or those who have had an abnormal maternal serum (blood) screening test indicating an increased risk for a chromosomal abnormality or neural tube defect. In one instance, a needle, e.g., a long, thin, hollow needle, can be used with ultrasound guide through your abdomen, into the uterus and the amniotic sac. A predetermined amount of amniotic fluid, e.g. one ounce, can be drawn into a syringe.

In some instances, the mammalian stem cell herein can be obtained from blastomere biopsy during preimplantation genetic diagnosis (PGD), e.g., in conjunction with reproductive therapies such as in vitro fertilization (IVF). In one instance, the cells herein can be produced by methods for biopsy of a blastocyst, wherein the remainder of the blastocyst is implanted and results in a pregnancy and later in a live birth, e.g., the zona pellucida is removed from the blastocyst and then the blastocyst is biopsied.

In some instances, a mammalian stem cell herein can be obtained from prenatal chorionic villus sampling (CVS). In one instance, CVS can be a prenatal test that involves taking a sample of tissue from the placenta to test for chromosomal abnormalities and certain other genetic problems. In one instance, CVS can be performed between the 10th and 12th weeks of pregnancy. In one instance, the CVS procedure is transcervical, e.g., a catheter is inserted through the cervix into the placenta to obtain the tissue sample. In one instance, the CVS procedure is transabdominal, e.g., a needle is inserted through the abdomen and uterus into the placenta to obtain the tissue sample.

In some instances, the mammalian stem cell herein can be isolated from first trimester chorionic villous sampling (e.g., 8⁺³ to 12⁺⁰ weeks gestational age) or term placenta from caesarean section deliveries. The chorionic tissue can be separated from the amnion, minced, and/or enzymatically digested (e.g., with 0.05% trypsin EDTA, e.g., for 20 min). Cells are subsequently centrifuged (e.g., at 1500 rpm, e.g., for 5 min), counted, and/or replated (e.g., 104 cells per cm²) in a medium (e.g., Dulbecco's modified Eagle's medium+10% fetal bovine serum). In one instance, isolated cells can be plastic adherent. In one instance, the cells can be used at passage 4-8.

In some instances, chorionic villi can be obtained from the fallopian tubes of un-ruptured pre-implantation embryos in women with ectopic pregnancy (e.g., gestational age: 5-8 weeks or 6-8 weeks, or 4-8 weeks post fertilization). Tiny villous tissues can be well-minced in a suitable medium (e.g., serum-free α-MEM) and identified under microscopy followed by trypsinization (e.g., with 0.025% trypsin/EDTA) for a period of time (e.g., 15 min) and by adding a medium (e.g., α-MEM containing 10% FBS) to halt the reaction. Adherent cells can be obtained and cultured in a suitable condition (e.g., in conditioned α-MEM, 10% FBS, and 1% penicillin-streptomycin at 37° C. in 5% CO₂). After two passages, the level of hCG can become undetectable measured by a commercial kit (e.g., DAKO®, Carpinteria, Calif.).

### Kits/Articles of Manufacture

Disclosed herein are kits and articles of manufacture for use with one or more methods and compositions described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In some instances, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of use.

For example, the container(s) include cells, optionally in a composition as disclosed herein. Such kits optionally include an identifying description or label or instructions relating to its use in the methods described herein.

A kit typically includes labels listing contents and/or instructions for use, and package inserts with instructions for use. A set of instructions will also typically be included.

In some instances, a label is on or associated with the container. In some instances, a label is on a container when letters, numbers or other characters forming the label are attached, molded or etched into the container itself; a label is associated with a container when it is present within a receptacle or carrier that also holds the container, e.g., as a package insert. In some instances, a label is used to indicate that the contents are to be used for a specific therapeutic application. The label also indicates directions for use of the contents, such as in the methods described herein.

### EXAMPLES

The Examples below are non-limiting and merely representative of various aspects and features of the present inventions.

### Example 1

### MPSCs Reached 89 Population Doublings

Non-embryonic stem cells for example trophoblast stem cells came from human donors as source cells. Several culture media were tested to culture the cells to grow into mortal pluripotent stem cells (MPSCs), as shown in Table 1 below.

TABLE 1
Culture media for growing MPSCs.

| Medium | Supplements | Coating |
|---|---|---|
| MEM Alpha + GlutaMAX | Stemulate 10% Human Platelet | — |
| (Gibco 32571-036) | Lysate Cell Culture Media | |
| | Supplement | |
| | (Cook Regentec PL-NH-100) | |
| MEM Alpha + GlutaMAX | Stemulate 10% Human Platelet | CELLBIND ® surface |
| (Gibco 32571-036) | Lysate Cell Culture Media | (Corning) |
| | Supplement | |
| | (Cook Regentec PL-NH-100) | |
| MEM Alpha + GlutaMAX | Stemulate 10% Human Platelet | Laminin |
| (Gibco 32571-036) | Lysate Cell Culture Media | (0.5 μg/cm²) |
| | Supplement | |
| | (Cook Regentec PL-NH-100) | |
| MESENCULT ®-ACF Plus | MESENCULT ®-ACF Plus | Cell Attachment Substrate |
| (StemCell Technologies, 05448) | 500× Supplement, Glutamine | (CAS) |
| | 2 mM | |
| STEMPRO ® MSC SFM | STEMPRO ® MSC SFM | Fibronectin |
| XenoFree | XenoFree Supplement | (0.4 μg/cm²) |
| (Gibco, A1067501) | Glutamine 2 mM | |
| RoosterNourish-MSC-XF | RoosterBooster Supplement | CELLBIND ® surface |
| (RoosterBio, KT-016) | | (Corning) |
| Prime XV MSC Expansion | — | Fibronectin |
| XSFM | | (0.4 μg/cm²) |
| (Irvine Scientific, 91149) | | |
| Prime XV NK cell CDM | rhIL2-ACF 550 IU/ML | — |
| (Irvine Scientific, 91215) | | |

Lowering seeding density from 10,000 to 2,000-4,000 cells/cm² improved the number of population doublings for MPSCs. 3-day subcultures of cells seeded at densities between 3000-5000 cells/cm² generate similar numbers of PD. Alternating 3-day/4-day subcultures of cells seeded at 4000/3000 cells/cm² generates similar numbers of PD as 3-day subcultures at 4000 cells/cm² for earlier passages. Culture environment can be 21% O₂/% CO₂, or 2% O₂/5% CO₂/93% N₂.

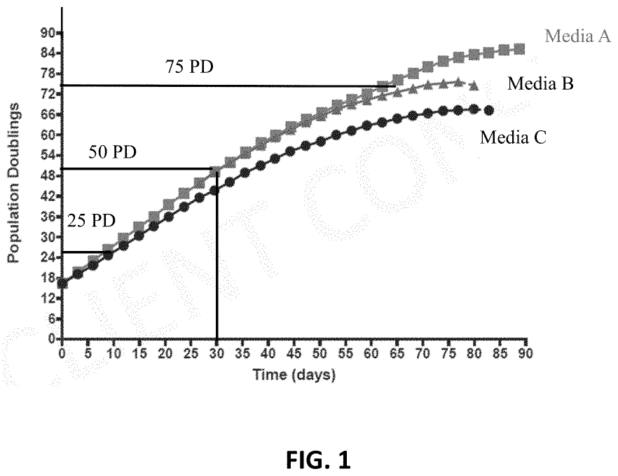

Some of the growth results over 90 days, i.e., 30 passages of 3-day-subcultures, are displayed in FIG. 1, which is a line chart showing that 3-day-subculture growth curves of MPSCs measured by population doublings (PD) in a time frame of 90 days. The MPSCs reached up to 25 PD by about 12 days, up to 50 PD by about 30 days, up to 75 PD by about 63 days, up to 89 PD by about 90 days.

MPSCs possess an extended population doubling capacity of ˜70-80 doublings with a doubling time of ˜27 hours when cultured in xeno-free media. It takes about averagely 27 hours for the cells to double in population, which can be calculated with an equation T=td/log 2[(2−y)/(1−y)], where T is the duration of the cell cycle, td is the average time of duplication of cell number, and y is proportion of cells in G0 phase.

This extended doubling capacity make MPSCs an ideal population of cells for expansion at industrial scale, eliminating the need for repeated isolation from a donor or biological source. The large batch size potential of MPSCs would generate sufficient MPSCs from a single derivation, streamlining therapeutic development and manufacturing and accelerating the realization of stem cell based therapeutics in the clinic by reducing product variability associated with multiple banks and the costs of manufacturing and releasing comparable product from different donors.

### Example 2

### MPSCs have no Chromosomal Aberrations

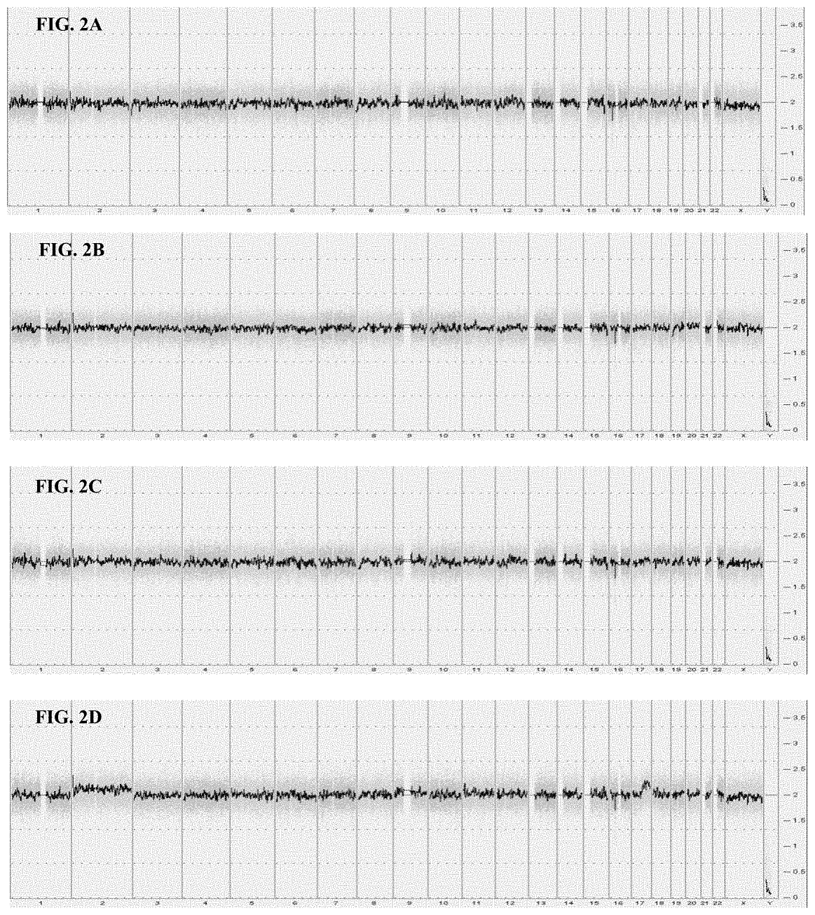

FIGS. 2A-2D show a whole genome view of a KARYOSTAT™ from four different MPSC samples from different population doublings. A KARYOSTAT™ assay can allow for digital visualization of chromosome aberrations. The size of structural aberration that can be detected is about >2 Mb (megabase) for chromosomal gains and about >1 Mb for chromosomal losses. Genomic DNA was purified from cells and the genomic DNA was added to the GENECHIP® for the KARYOTATE™. The GENECCHIP® can determine copy number variants of chromosomes. FIGS. 2A-2D show the whole genome view that displays all somatic and sex chromosomes in one frame. FIG. 2A, is a MPSC sample from 16.5 population doublings, FIG. 2B, is a MPSC sample from 44.5 population doublings, FIG. 2C is a MPSC sample from 62.6 population doublings, and FIG. 2D is a MPSC sample from 71.5 population doublings. The smooth signal plot (right y-axis) is the smoothing of the log 2 ratios which depict the signal intensities of probes on the microarray. A value of 2 can represent a normal copy number state (CN=2). A value of 3 can represent a chromosomal gain (CN=3). A value of 1 can represent a chromosomal loss (CN=1). The gray signal indicates the raw signal for each individual chromosome probe, while the black signal represents the normalized probe signal which is used to identify copy number and aberrations (if any). No observable chromosomal aberrations were found in FIGS. 2A-D. The MPSC cells can go through multiple population doublings without chromosomal aberrations. For example, a monoclone population could be expanded and then frozen for future use. Once the cells are grown from the frozen stock cultures of monoclones they can be expanded without substantial chromosomal aberrations. The chromosome stability of MPCSs provides another advantage over human embryonic stem cells and iPSCs which often show genetic abnormalities or mutations associated with immortality.

### Example 3

### Characterization of MPSCs by Expressing Specific Molecular Biomarkers

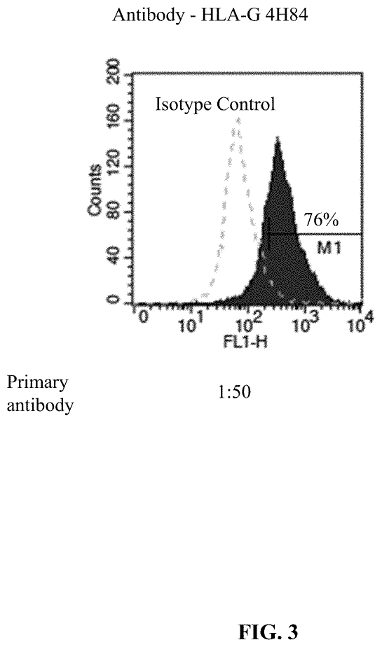

MPSCs express an immune-privilege marker HLA-G. Unlike adult or post-natal human mesenchymal stromal cells, MPSCs herein express the human leukocyte antigen-G (HLA-G), a major histocompatibility complex class I antigen exclusive to the placenta that binds to HLA-G receptors on leukocytes to suppress immune function via numerous mechanisms, including triggering apoptosis in activated T cells, modulating the activity of Natural Killer cells and dendritic cells, and inhibiting T-cell proliferation. Referring to FIG. 3, this figure shows MPSCs stained with the primary antibody 4H84. For FIG. 3, the MPSCs were harvested from culture in MESENCULT®-ACF Plus Medium. Cells were resuspended in flow cytometry wash buffer (Gibco DPBS, substantially without calcium chloride or magnesium chloride, about 2% fetal bovine serum and about 0.1% sodium azide) and aliquoted from about 0.25-0.5×10⁶ cells per sample into a flow cytometry tube and the cells were centrifuged. The cells were fixed with a 4% paraformaldehyde solution for about 15 min at room temperature. In some instances, after fixation the cells were permeabilized with about 500 μl (microliters) of cold Perm Buffer III (BD Biosciences) and then the cells were incubated on ice. The permeabilization permitted intracellular material to be stained. After the incubation, the cells were washed with flow cytometry wash buffer, centrifuged and resuspended in flow cytometry staining buffer (R&D Systems). The primary antibody staining occurred when a dilution of an HLA-G primary antibody (e.g. 4H84 antibody) was added to the cells and incubated at room temperature. The cells were washed several times with flow cytometry wash buffer. After the primary antibody had been bound to the cells, a secondary antibody was added. The secondary antibody procedure took place in the dark. The secondary antibody was added at a dilution of about 1:2000 into flow cytometry staining buffer. The cells were resuspended in about 100 μl of diluted secondary antibody solution and incubated for about 30 minutes. The cells were washed several times in flow cytometry wash buffer. After the cells were washed, the cells were resuspended in flow cytometry staining buffer to a concentration of about 0.5×10⁶ cells. After the cells were resuspended, the cells were sampled by flow cytometry. The primary antibody MEM-G/11 can recognize the HLA-G1 isoform which is membrane bound. The primary antibody 4H84 antibody can recognize the alpha domain of 7 isoforms of HLA-G. FIG. 3 shows MPSCs permeabilized and stained with the primary antibody HLA-G 4H84. The staining shows the presence of HLA-G isoforms in or on MPSCs. The staining shows about 76% of the cells in a 1:50 primary antibody dilution were positive compared to an isotype control. FIG. 4 shows the cells stained with the primary antibody 4H84 and the primary isotype control antibody mouse IgG1. The cells show limited staining with the IgG1 antibody and 99.64% of events were stained with the 4H84 antibody, which indicates the antibody is specific to MPSCs. The expression of HLA-G in MPSCs as shown in FIG. 3, may allow the cells to have access to immune privileged sites, for example, a fetus. The MPSCs herein have shown human MSC phenotype and morphology by expressing the characteristic markers as measured by fluorescence activated cell sorting (FACS), see Table 2 below.

TABLE 2
Expression of the markers showing mesenchymal stromal cell - like phenotype

| | NEGATIVE | | | POSITIVE MARKERS | | | | | |
|---|---|---|---|---|---|---|---|---|---|
| | MARKERS | | | | | | | | HLA- |
| | CD19 | CD45 | HLA-DR | CD44 | CD90 | CD105 | CD146 | CD166 | A, B, C |
| PD | (−) | (−) | (−) | (+) | (+) | (+) | (+) | (+) | (+) |
| 19.8 | 99.9 | 98.6 | 99.6 | 99.7 | 99.8 | 96.6 | 92.1 | 99.9 | 100 |
| 41.4 | 99.9 | 99.6 | 99.8 | 98.4 | 99.8 | 92.7 | 91.0 | 99.9 | 99.8 |
| 44.6 | 99.9 | 99.7 | 99.7 | 97.7 | 99.9 | 94.9 | 91.7 | 100 | 99.9 |
| 41.7 | 99.2 | 98.5 | 99.2 | 96.4 | 99.8 | 93.2 | 90.5 | 100 | 99.9 |

The MPSCs herein may provide a solution as an alternative to mesenchymal stromal cells (MSCs). Human MSCs exert immunosuppressive effects, demonstrate tri-lineage differentiation in vitro, and have been safely delivered to patients for a variety of indications, and have been approved to treat niche indications such as autoimmune-mediated perianal fistulas and Graft versus Host disease. However, widespread adoption of MSC-based therapies has been hindered by an inability to manufacture large batches of MSCs due to a population doubling limit of about 30-40 doublings before reaching cellular senescence. The MPSCs herein have shown natural killer cell phenotypes as measured by FACS, see Table 3 below.

TABLE 3
Expression of the markers showing
natural killer cell phenotype

| | CD3 | CD56 | CD16 |
|---|---|---|---|
| PD | (−) | (+) | (+) |
| 19.8 | 99.4 | 56.4 | 2.0 |
| 41.4 | 99.7 | 23.3 | 0.5 |
| 44.6 | 99.6 | 22.4 | 0.8 |
| 41.7 | 99.5 | 19.3 | 1.0 |

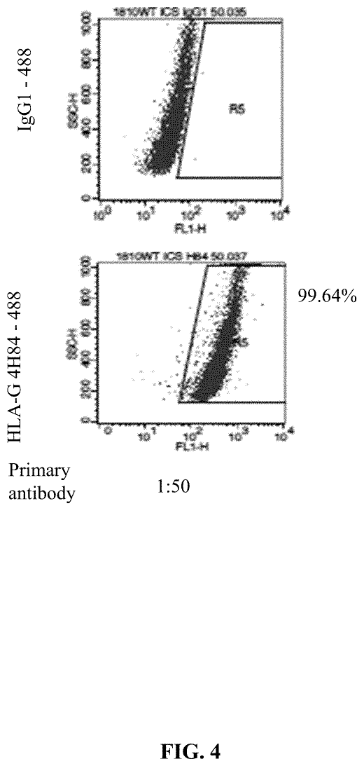

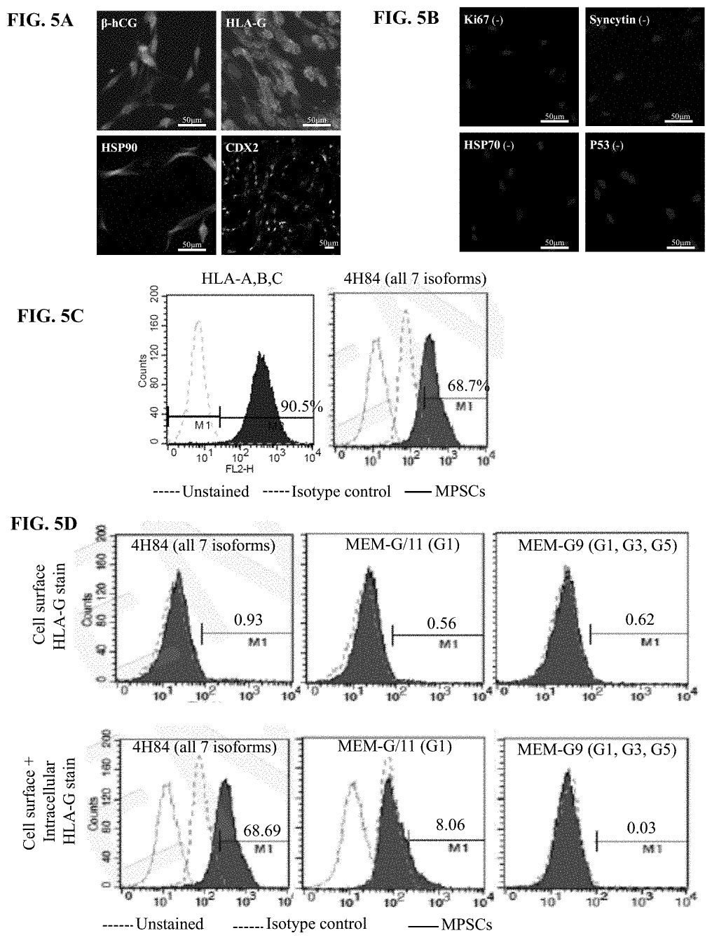

MPSCs expressed a variety of cell biomarkers, including β-hCG, HLA-G, heat shock protein 90 (HSP90), and CDX2 immunocytochemically (FIG. 5A). However, MPSCs did not express proliferation marker Ki-67, HSP70, tumor suppressor p53, and cell-cell fusion protein Syncytin (FIG. 5B), supporting the concept that MPSCs stand at the first position of TE-differentiated trophoblasts. Specifically, MPSCs expressed HLA-A,B,C and surface and intracellular HLA-G by flow cytometry analysis (FACS) using different antibodies (FIG. 5C; FIG. 5D). However, they did not express HLA-DR.

FIG. 5D are representative FACS images of HLA-G isoforms in MPSCs. Very few of all 7 isoforms detected at cell surface (upper left column) but 68.7% of all HLA-G 7 isoforms detected in permeabolised MPSCs (left lower column) by using Ab 4H84. While few of HLA-G G1 at cell surface (upper middle column) but 8.1% of HLA-G G1 (upper middle column) detected by Ab MEM-G/11. Similar few of HLA-G G1, G3, G5 detected at cell surface (upper right column) but none of HLA-G detected by Ab MEM-G9.

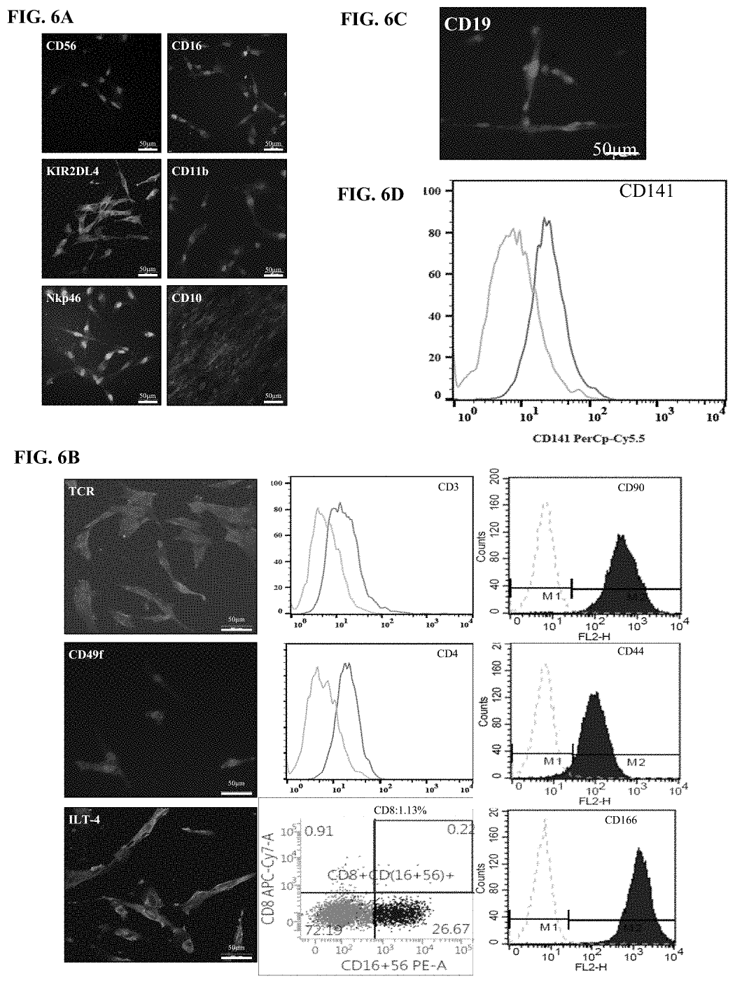

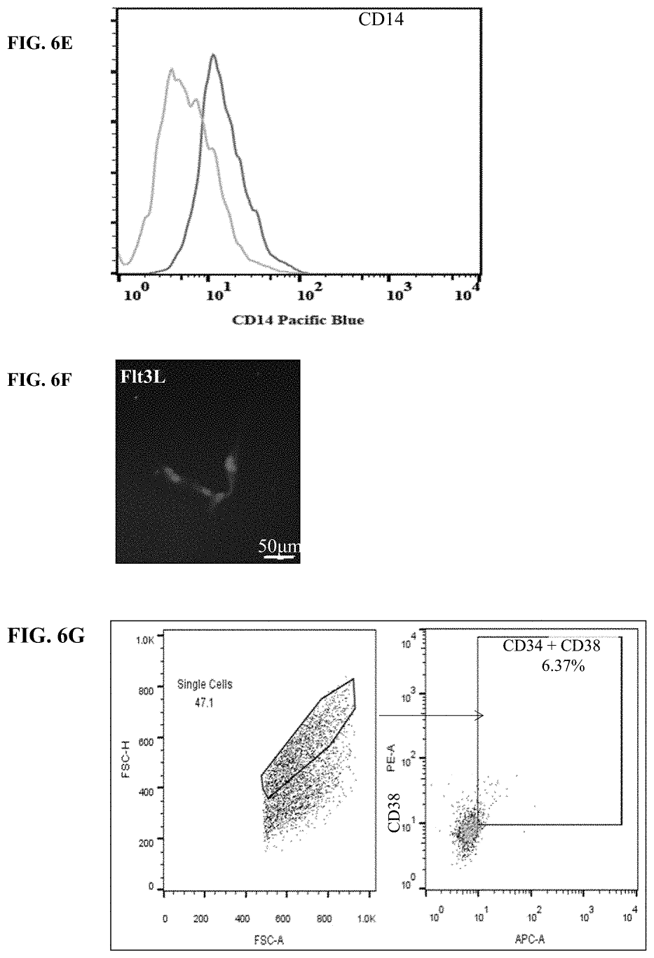

Human MPSCs Exhibit Immune Cell-Associated Biomarkers. The cells were characterized with immunocytochemistry and FACS analysis. The results showed that MPSCs expressed a variety of biomarkers associated with immune cells, including: cluster of differentiation (CD)56, CD16^{dim}, inhibitory receptor KIR2DL4, CD11b, activating receptor NKp46, and CD10 of NK cells (FIG. 6A); TCR, CD49f, ILT-4, CD3, CD4, CD8, CD44, CD90/Thy-1, CD44, and CD166 of T cells (FIG. 6B); CD19 and CD141 of dendritic cells (FIGS. 6C and 6D); CD14 of macrophages (FIG. 6E); Flt3L (FIG. 6F) and CD34 of hematopoietic stem cells (FIG. 6G); and CD38 of lymphocytes (FIG. 6G). Subsequently, the expression of those biomarkers in MPSCs was analyzed with 8 independent cell lines, showing a similar pattern of NK and T cell biomarkers, wherein (CD16+CD56)⁺ cells and CD107(+) cells showed the highest expression in the MPSCs. Biomarkers of NK cells and T cells occupy the most immune cells in MPSCs ,while CD107(+)CD(16+56)(+) cells and CD8(+)CD(16+56)(+) cells occupied the most cell populations in MPSCs by FACS analysis.

### Example 4

### A Significant Portion of MPSCs are Monoclonal

MPSC monoclones were obtained from a MPSC culture. MPSCs were grown placed on an inverted microscope to record the cell type. The old medium was removed and cells were washed with sterile PBS. TRYPLE® solution was added to the MPSC culture. The cells were incubated at 37° C., 5% CO₂ for about 6 minutes. After incubation, the cells were separated. Culture media was added to the cells to stop the TRYPLE® reaction. The cells were collected, and a cell counter was used to calculate the number of cells. About 200 cells were removed and placed in a centrifuge tube. The media was replenished, and the cells were divided into 96 well plates with a multichannel pipette. The 96 well plates were grown at 37° C., 5% CO₂, and incubated for about 14 days. The media was changed every 2-3 days during this process. The old medium was washed and a TRYPLE® solution was added to the cells. After a short incubation, culture media was added to stop the TRYPLE® reaction, and the cells were moved to a 6 well plate and grown at 37° C. and 5% CO₂. The culture media was changed every 2-3 days until they were subcultured into a 100 mm dish and grown at 37° C. and 5% CO₂. The media was changed every 2-3 days. The monoclone cells were frozen when they reach 80-95% thickness. From donor ectopic tissue, the cells were expanded as wild-type passages with mixed cells or they were expanded into monoclones. Monoclones were expanded to provide multiple doses. For example, for every 1 million cells derived from donor ectopic tissue, about 125,000 monoclones can be cultured. Each monoclone has the potential for 7×10²⁸ cells. At 100 million cells per dose, each monoclone can make 7×10²⁰ doses. Total potential from each ectopic tissue collected: 125,000×7×10²⁰ doses=8.8×10²⁵ doses. Each MPSC monoclone can support a complete product cycle.

Every vial of 1M cells can potentially net about 130,000 monoclones. See Table 4 below.

TABLE 4
Monoclone percentages among the MPSCs

| Cell | Cells | Monoclones | |
|---|---|---|---|
| lines | Seeded | Established | % |
| I | 1600 | 203 | 12.7% |
| II | 800 | 119 | 14.9% |
| III | 400 | 50 | 12.5% |
| Average | | | 13.3% |

### Example 5

### MPCSs are Pathogen-Free

Regardless of whether source cells were infected or free from pathogen, the human MPCS cells obtained herein were pathogen-free. Nine cell lines of MPSCs were tested, and all of them were found negative for all 25 pathogens in the h-IMPACT I panel. PCR evaluation was done to detect Corynebacterium bovis, Corynebacterium sp. (HAC2), EBV, HAdV, Hantaan, HCMV, Hepatitis A, Hepatitis B, Hepatitis C, HHV 6, HHV 8, HIV1, HIV2, HPV16, HPV18, HSV 1, HSV 2, HTLV 1, HTLV2, LCMV, Mycoplasma sp., Seoul, Sin Nombre, Treponema pallidum, VZV. The results were shown in Table 5 below.

TABLE 5
PCR Evaluation of MPSC cell lines

| Cell line No. | 1 | 2 | 3 | 4 | 5 | 6 | 7 | 8 | 9 |
|---|---|---|---|---|---|---|---|---|---|
| EBV | − | − | − | − | − | − | − | − | − |
| HAdV | − | − | − | − | − | − | − | − | − |
| HCMV | − | − | − | − | − | − | − | − | − |
| Hepatitis A | − | − | − | − | − | − | − | − | − |
| Hepatitis B | − | − | − | − | − | − | − | − | − |
| Hepatitis C | − | − | − | − | − | − | − | − | − |
| HHV 6 | − | − | − | − | − | − | − | − | − |
| HHV 8 | − | − | − | − | − | − | − | − | − |
| HIV1 | − | − | − | − | − | − | − | − | − |
| HIV2 | − | − | − | − | − | − | − | − | − |
| HPV16 | − | − | − | − | − | − | − | − | − |
| HPV18 | − | − | − | − | − | − | − | − | − |
| HSV 1 | − | − | − | − | − | − | − | − | − |
| HSV 2 | − | − | − | − | − | − | − | − | − |
| HTLV 1 | − | − | − | − | − | − | − | − | − |
| HTLV 2 | − | − | − | − | − | − | − | − | − |
| VZV | − | − | − | − | − | − | − | − | − |
| Corynebacterium bovis | − | − | − | − | − | − | − | − | − |
| Corynebacterium sp. (HAC2) | − | − | − | − | − | − | − | − | − |
| Hantaan | − | − | − | − | − | − | − | − | − |
| LCMV | − | − | − | − | − | − | − | − | − |
| Mycoplasma sp. | − | − | − | − | − | − | − | − | − |
| Seoul | − | − | − | − | − | − | − | − | − |
| Sin Nombre | − | − | − | − | − | − | − | − | − |
| Treponema pallidum | − | − | − | − | − | − | − | − | − |

Legend: + = positive, − = negative, id:id = pooled sample range, id + id + id = non-range pooled sample, NT or blank = no test performed, wps = weak positive, XX = Testing in progress.

While some embodiments have been shown and described herein, such embodiments are provided by way of example only. Numerous variations, changes, and substitutions can occur without departing from the inventions. It should be understood that various alternatives to the embodiments of the inventions described herein can be employed in practicing the inventions.

What is claimed is:

1. A population of mortal pluripotent stem cells (MPSCs), wherein the population of MPSCs express HLA-G and insulin, and wherein the population of MPSCs are capable of reaching up to about 89-100 population doublings within 90 days from a start of culturing the MPSCs.

2. The population of MPSCs of claim 1, wherein the population of MPSCs are capable of reaching: about 25-30 population doublings within 12 days, about 50-55 population doublings within 30 days, or about 75-80 population doublings within 63 days, from the start of culturing the MPSCs.

3. The population of MPSCs of claim 1, wherein the population of MPSCs are capable of doubling in about 22-27 hours.

4. The population of MPSCs of claim 3, wherein the population of MPSCs are capable of doubling in about 25 hours.

5. The population of MPSCs of claim 1, wherein the population of MPSCs are free from a pathogen.

6. The population of MPSCs of claim 5, wherein the pathogen is a bacterium.

7. The population of MPSCs of claim 5, wherein the pathogen is a virus.

8. The population of MPSCs of claim 7, wherein the virus is a cytomegalovirus.

9. The population of MPSCs of claim 5, wherein the pathogen is selected from the group consisting of EBV (Epstein-Barr virus), HAdV (human adenovirus), HCMV (human cytomegalovirus), Hepatitis A, Hepatitis B, Hepatitis C, HHV 6 (human herpes virus 6), HHV 8 (human herpes virus 8), HIV1 (human immunodeficiency virus 1), HIV2 (human immunodeficiency virus 2), HPV (human papillomavirus), HPV16, HPV18, HSV 1 (herpes simplex 1), HSV 2 (herpes simplex 2), HTLV 1 (human T-lymphotropic virus 1), HTLV 2 (human T-lymphotropic virus 2), VZV (varicella virus), Corynebacterium bovis, Corynebacterium sp. (HAC2), Hantavirus comprising Hantaan, Seoul, or Sin Nombre, LCMV (lymphocytic choriomeningitis virus), Mycoplasma sp., Treponema pallidum, and any combination thereof.

10. The population of MPSCs of claim 1, wherein the population of the MPSCs further express b-HCG, HSP90, CDX2, FGFR1, pAKT, pCREB1, HLA-A, HLA-B, HLA-C, or a combination thereof.

11. The population of MPSCs of claim 1, wherein the population of the MPSCs further express KIR2DL4, Flt3L, NKp46, TCR, ILT-4, CD49f, CD3, CD4, CD8, CD10, CD11b, CD14, CD16, CD19, CD34, CD38, CD44, CD56, CD90/Thy-1, CD105, CD141, CD146, CD166, CD107a, or a combination thereof.

12. The population of MPSCs of claim 1, wherein the population of the MPSCs further express IL-6, IL-8, MCP-1, CLXL2, PDGF-AA, VEGF, PAI-1, IL-10, or a combination thereof.

13. The population of MPSCs of claim 1, wherein at least some of the MPSCs do not express Ki-67, HSP70, p53, Syncytin, or a combination thereof.

14. The population of MPSCs of claim 1, wherein the population of the MPSCs express CD44, CD90, CD105, CD146, CD166, HLA-A, HLA-B, HLA-C, or a combination thereof.

15. The population of MPSCs of claim 1, wherein at least some of the MPSCs do not express CD19, CD45, HLA-DR, or a combination thereof.

16. The population of MPSCs of claim 15, wherein more than 96% of the MPSCs do not express CD19, CD45, HLA-DR, or a combination thereof.

17. The population of MPSCs of claim 1, wherein the population of the MPSCs express CD16, CD56, or a combination thereof.

18. The population of MPSCs of claim 1, wherein at least some of the MPSCs do not express CD3.

19. The population of MPSCs of claim 18, wherein more than 96% of the MPSCs do not express CD3.

20. The population of MPSCs of claim 1, wherein at least 65% of the population of the MPSCs express the HLA-G.

21. The population of MPSCs of claim 20, wherein the HLA-G comprises HLA-G1, HLA-G2, HLA-G3, HLA-G4, HLA-G5, HLA-G6, HLA-G7, or any combination thereof.

22. The population of MPSCs of claim 21, wherein the HLA-G comprises HLA-G2, HLA-G4, HLAG-6, HLA-G7, or any combination thereof.

23. The population of MPSCs of claim 22, wherein the HLA-G comprises HLAG-6, HLA-G7, or a combination thereof.

24. The population of MPSCs of claim 21, wherein less than 15% of the population of the MPSCs express HLA-G1.

25. The population of MPSCs of claim 1, wherein at least 10% of the population of MPSCs are monoclonal.

26. The population of MPSCs of claim 25, wherein from about 13% to about 15% of the population of MPSCs are monoclonal.

27. The population of MPSCs of claim 1, wherein at least 1×10⁶ MPSCs are present in the population.

28. The population of MPSCs of claim 1, wherein the MPSCs have a stable karyotype as measured by an array-based whole-genome assay.

29. The population of MPSCs of claim 28, wherein the MPSCs experience no chromosomal aberration from population doublings as measured by the array-based whole-genome assay.

30. The population of MPSCs of claim 28, wherein the MPSCs experience no substantial chromosomal aberration from freezing and thawing as measured by the array-based whole-genome assay.

31. A method of growing the population of MPSCs, comprising seeding a subculture of the MPSCs at a density of from about 1,000 to about 5,000 cells/cm² in a culture medium.

32. The method of claim 31, wherein the population of MPSCs express HLA-G and insulin.

33. The method of claim 31, wherein the culture medium is free from an animal component.

34. The method of claim 33, wherein the culture medium is free from a serum.

35. The method of claim 34, wherein the culture medium is free from a fetal bovine serum.

36. The method of claim 31, wherein the subculture comprises a 3-day subculture.

37. The method of claim 31, wherein the subculture comprises a 4-day subculture.

38. The method of claim 31, wherein the subculture of the MPSCs is at a density of from about 2,000 to about 4,000 cells/cm².

39. A method of producing cells, comprising contacting the population of MPSCs of claim 1 with an inducing agent.

40. The method of claim 39, wherein the cells are ectodermal cells.

41. The method of claim 39, wherein the cells are mesodermal cells.

42. The method of claim 39, wherein the cells are endodermal cells.

43. The method of claim 39, wherein the cells are pancreatic cells or pancreatic progenitor cells.

44. The method of claim 43, wherein the inducing agent comprises bFGF (basic fibroblast growth factor).

45. The method of claim 44, wherein the inducing agent further comprises 2-mercaptoethanol and nicotinamide.

46. The method of claim 39, wherein the cells are neural cells or neural progenitor cells.

47. The method of claim 46, wherein the inducing agent comprises retinoic acid.

48. The method of claim 39, wherein the cells are hepatic cells or hepatic progenitor cells.

49. The method of claim 48, wherein the inducing agent comprises a fibroblast growth factor (FGF), a steroid, and a cytokine.

50. The method of claim 39, wherein the cells are natural killer cells and the inducing agent comprises an FGF.
